(19) United States
(12) Patent Application Publication
(10) Pub. No.: US 2018/0016538 A1
(43) Pub. Date: Jan. 18, 2018

(54) APPARATUS, SYSTEMS AND METHODS FOR PROGRAMMABLE TISSUE CULTURE ILLUMINATION

(71) Applicant: Regents of the University of California, San Francisco, CA (US)

(72) Inventors: Lukasz Bugaj, San Francisco, CA (US); Wendell Lim, San Francisco, CA (US)

(21) Appl. No.: 15/651,628

(22) Filed: Jul. 17, 2017

Related U.S. Application Data
(60) Provisional application No. 62/362,768, filed on Jul. 15, 2016.

(51) Int. Cl. C12M 1/00; C12M 1/42; C12M 1/12

(52) U.S. Cl. CPC C12M 31/10; C12M 41/06; C12M 23/38; C12M 25/06; C12M 35/02

(57) ABSTRACT

The disclosed apparatus, systems and methods relate to an illumination opto-plate configured to specifically light the wells of a culture plate.

### CROSS REFERENCE TO RELATED APPLICATIONS

This application claims priority to the U.S. Provisional Application No. 62/362,768 filed Jul. 15, 2016 and entitled “Apparatus, Systems, and Methods for Programmable Tissue Culture Illumination” which is hereby incorporated by reference in its entirety under 35 U.S.C. §119(e).

### GOVERNMENT SUPPORT

This invention was made with government support under Grant Nos. R11 GM096164 and R01 GM055040, awarded by the National Institutes of Health. The government has certain rights in the invention.

### TECHNICAL FIELD

This technology relates to devices, systems and methods for illumination of tissue culture plates. This has implications in several fields of study, including optogenetics.

### BACKGROUND

The disclosure relates to devices, systems and methods for illuminating plates, such as tissue culture plates. The ability to specifically apply light to living cells in culture presents several possibilities in various fields, including optogenetics. For example, the use of specific colors (or bandwidths) at specific times to illuminate living cells in cell culture can allow control of those cells using light.

Methods to control protein activity in living cells have emerged recently and have since spawned a new field of research called optogenetics. Despite the power of these tools, there exists a lack of commercial hardware to illuminate biological samples in a manner compatible with standard cell culture. Instead, researchers often use light sources designed for microscopy or large LED panels that illuminate a large area, such as an incubator. These are low-throughput solutions, and in the case of the LED panel, also low resolution solutions. There is a need in the art for improved optogenetics illumination devices, systems and methods.

### BRIEF SUMMARY

Discussed herein are various devices, systems and methods relating to illumination of individual wells of standard cell culture formats with up to three distinct LED colors. Illumination intensity and timing of each LED is fully programmable, allowing independent illumination profiles in each well with no crossover illumination between wells.

The disclosed illumination device has numerous applications, such as optogenetic activation, where precisely defined light activation of proteins in living samples is achieved in a high throughput manner. In further applications, the illumination device can be used in photoconversion, wherein discrete proteins are simultaneously photoconverted, such as in a 96-well plate. This has implications for imaging techniques that rely on photoconversion of fluorescent proteins. In additional applications, the illumination device can be used in photobiology investigating how biological samples such as plants, bacteria, and algae respond to light. Additional applications include drug screening to modulate optogenetic proteins in drug screening assays.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

The novel features are set forth with particularity in the claims that follow. A better understanding of the features and advantages will be obtained by reference to the following detailed description that sets forth illustrative embodiments and the accompanying drawings of which:

FIG. 1 shows an assembled illumination opto-plate, according to an exemplary embodiment.

FIG. 2A shows a detailed view of an illumination opto-plate, according to an exemplary embodiment.

FIG. 2B shows a close-up view of the opto-plate according to an exemplary embodiment.

FIGS. 3A-B show an exemplary implementation of the opto-plate.

FIGS. 4A-B show an exemplary implementation of the opto-plate.

FIG. 5 shows an exemplary implementation of the mounting points or headers for the microcontroller, the memory, the logic converter and the power jack.

FIG. 6A-B shows various opto-plate components installed on the plate, according to an exemplary embodiment.

FIG. 7A-B shows one possible embodiment of a 384-well plate adaptor.

FIG. 8 shows a 96-well lid and 384-well lid, according to an exemplary embodiment.

### DETAILED DESCRIPTION

The various embodiments disclosed or contemplated herein relate to devices, systems and methods for illumination of tissue culture plates. The various illumination device embodiments disclosed or contemplated herein allow independent illumination of the wells within standard cell culture plates, can be programmed to use variable timing and intensities, and can be mounted with LEDs of various wavelengths.

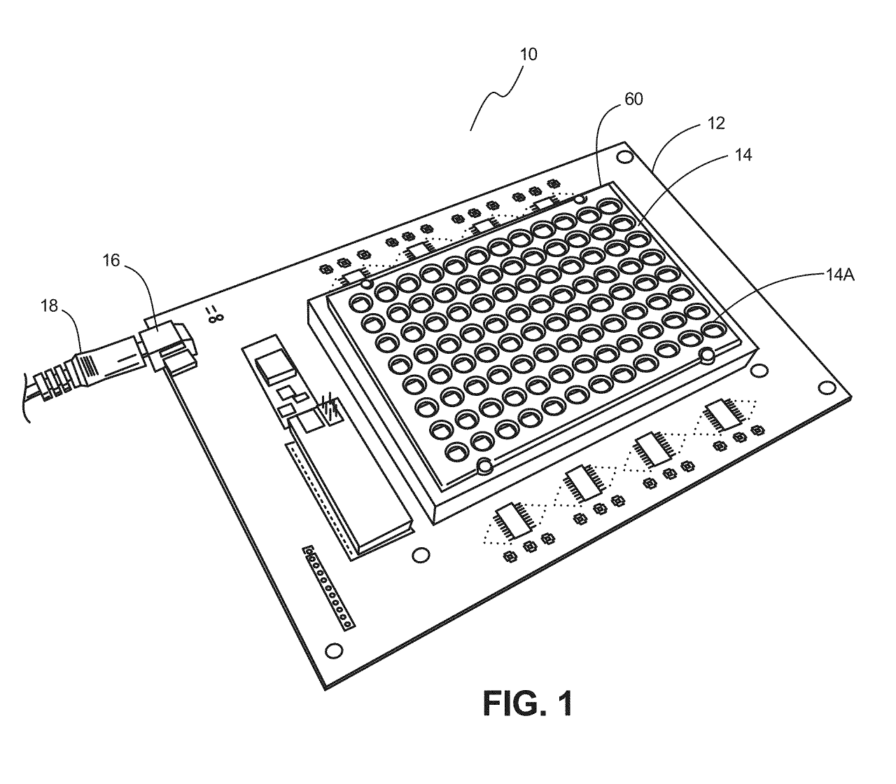

Turning to the drawings in greater detail, FIG. 1 is a perspective view of an assembled illumination opto-plate 10, according to one implementation. For purposes of this application, “opto-plate” is intended to mean any illumination device that can be used to illuminate a cell culture plate. It is understood that the opto-plate embodiments disclosed or contemplated herein are configured to receive any known cell culture plate (not shown) such that the plate is placed on top of any such embodiment. It is further understood that these known cell culture plates (not shown) must have wells that have a transparent or translucent bottom such that the illumination from any of the various opto-plate embodiments herein can enter the wells. Alternatively, such plates and/or well bottoms must allow light to pass through the underside of the plates/well bottoms in any known fashion to allow for illumination by any such opto-plate embodiment.

In this implementation of FIG. 1, the device 10 has a base 12 and a plate adaptor 14. It is understood that in the implementation of FIG. 1, the base 12 and plate adaptor 14 are configured to be used in conjunction with a 96-well plate as described above (such that the 96-well plate is positioned on top of the plate adaptor 14) (not shown), though configuration is possible for other common culture plates, such as 6-well, 12-well, 24-well, 48-well, 384-well plates and the like. In these various implementations, and as discussed above, the culture plate (not shown) is placed atop the adaptor 14 such that the various openings 60 defined in the adaptor 14 that correspond to the LED fixtures 20 discussed below are disposed below corresponding wells on the tissue culture plate (not shown). It is understood that the thin profile of the opto-plate 10 allows for versatile use, such as in an incubator or with a microscope.

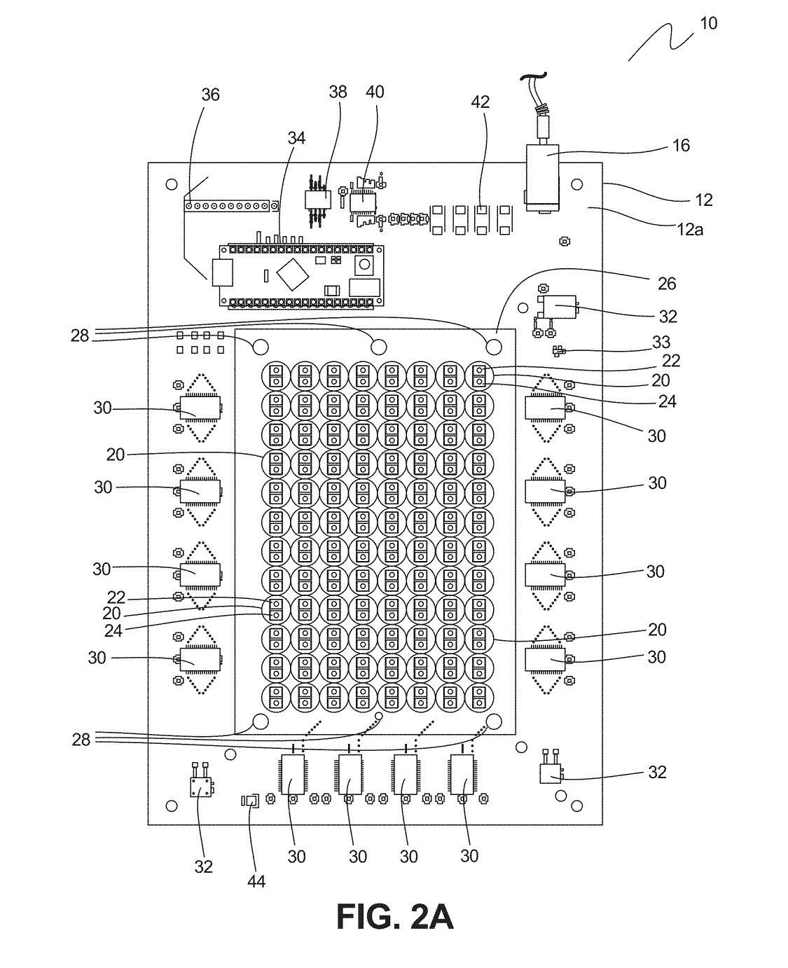

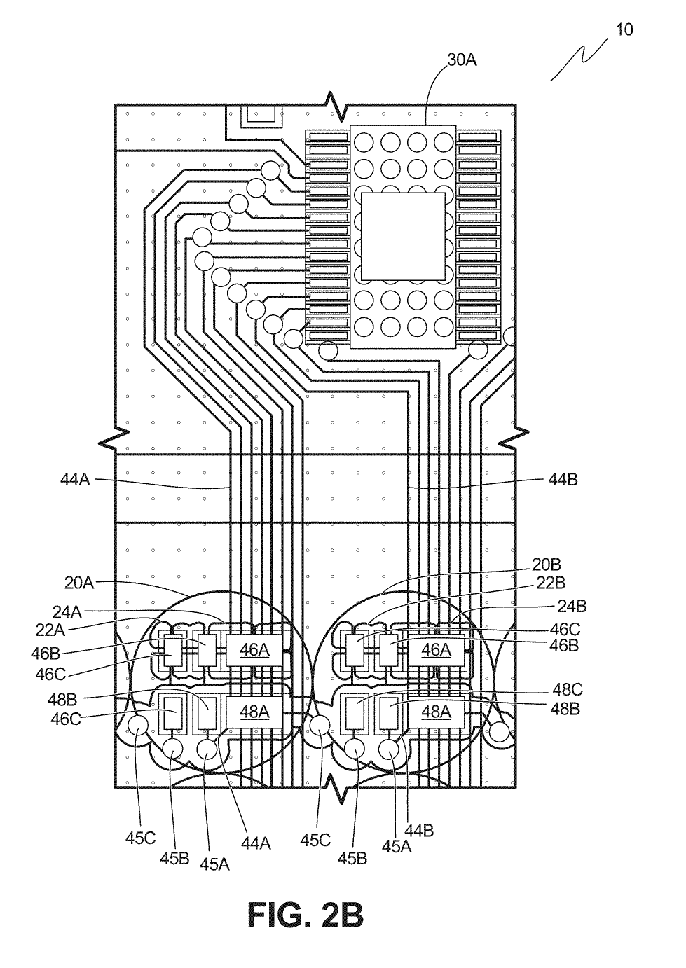

In alternate implementations, further applications known to one of skill in the art are possible. In various embodiments, the base 12 has a power jack 16 to receive electrical power from a power source 18 to power the various opto-plate components, such as the LED fixtures 20, as best shown in FIG. 2A and discussed below.

FIG. 2A is a detailed view of the opto-plate 10 with the adaptor 14 removed, according to one implementation. In this implementation, 96 LED fixtures 20 are disposed so as to be adjacent to and individually illuminate the wells of a 96-well plate (not shown) positioned atop the opto-plate 10 and be operated by a microcontroller 34. While LED fixtures 20 are used in this specific implementation, it is understood that other known lighting components used to illuminate well plates can be used in the various embodiments disclosed or contemplated herein.

According to various implementations, llumination intensity and timing of each of the LED fixtures 20 are fully programmable and controlled through the microcontroller 34, allowing for independent illumination profiles for each well. That is, each of the various LED fixtures 20 can be illuminated independently of any of the other fixtures 20, thereby allowing for independent illumination of any one or more of the corresponding wells of the plate (not shown). It is understood that several LED fixtures 20 are referenced in FIG. 2A, but for the clarity of the drawing, most of the 96 LED fixtures are not referenced.

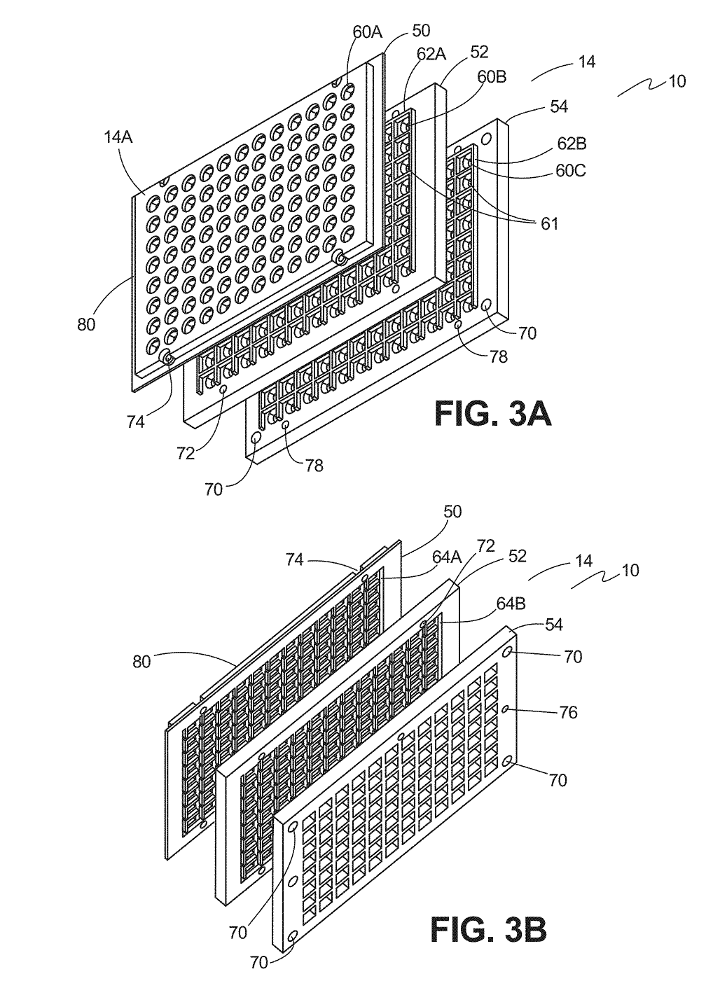

In this implementation of FIG. 2A, each of the LED fixtures 20 on this opto-plate 10 for a 96-well culture plate (not shown) has a 2-color LED 22 and a 1-color LED 24, such that each well of the culture plate can be illuminated with up to three colors, as would be understood in the art. It is understood that for 96-well implementations, there can be 96 such LED fixtures 20, each of which is individually operable, and that further implementations configured for alternate plate types and sizes will have corresponding numbers of LED fixtures 20 disposed on the face 12A of the base 12 within an LED board 26 to align with the adaptor 14 (shown in FIGS. 1 and 3A-B) and the subject well plate (not shown). In certain implementations, the LED board 26 has at least one opening 28 to allow attachment to the adaptor 14. That is, attachment devices or mechanisms such as pins, bolts, or any other such mechanisms can be used to couple the adaptor 14 to the board 26 via the one or more openings 28.

It is further understood that in implementations relating to smaller numbers of wells, such as a 6-well plate, other numbers of 2-color LED 22 and/or 1-color LED 24 lights may be used in each fixture 20 because the surface area and volume of the wells of a 6- or 12-well plate are larger than the wells of a 96-well plate. For example, in certain implementations of an opto-plate 10 for a 6-well culture plate (not shown), an individual lighting fixture 20 can have ten, twelve or more total lights made up of a combination of 2-color and 1-color LEDs, as desired.

In the implementation of FIG. 2A, groups of LED fixtures 20 can be driven by an LED driver 30, such as a 24-channel LED driver, which is shown further in relation to FIG. 2B. It is understood that in implementations for 96-well plates having a 2-color LED 22 and a 1-color LED 24 and using 24-channel LED drivers 30, there are 12 such drivers 30 in electrical and operable communication with the groups of LED fixtures 20, such that any individual 24-channel LED driver 30 is in operable communication with several 2-color LED 22 and 1-color LED 24 channels. It is understood that each color represents a channel, such that an individual driver could be connected to 12 2-color LEDs, 24 1-color LEDs, or any combination there between. The microcontroller 34 controls the LED drivers 30, allowing for the timing and intensity of each channel to be independently programmed. In these implementations, at least one PNP transistor 33 is in operational and electrical communication with the drivers 30 to control corresponding LED fixtures 20, as would be understood by the skilled artisan. A voltage regulator, such as a 5V voltage regulator 32, can also be provided.

Continuing with FIG. 2A, various implementations of the illumination device 10 have a microcontroller 34, such as, for example, an Arduino microcontroller, that can be mounted onto the board. In alternate implementations, an alternate microcontroller (not shown) can be connected to the LED fixtures by way of additional pin headers 36.

In various implementations, the microcontroller 34 is in operable communication with the LED fixtures 20 by way of the drivers 30 and transistor 33. In certain implementations, memory 38, such as RAM, and a logic converter 40, such as a 3V-5V logic converter, can also be disposed on the base 12 and in electrical and operable communication with the microcontroller 34 and/or LED fixtures 20 and other electrical components. In certain implementations, further known components, such as capacitors and the like, can also be operationally integrated by way of further surface-mount pads 42. It is understood that additional capacitors and resistors may also be operationally integrated into the electrical components to operate various electrical functions known to those of skill in the art.

It is understood that in these implementations, the use of a plug-in microcontroller 34 can obviate the need for a data cable connection during runtime, such that the opto-plate 10 can be pre-programmed and run independent of a computer connection. Further, through use of the microcontroller 34 and other components, in certain implementations the opto-plate 10 can have additional memory 38, such as RAM, that is user-programmable and can expand the memory capacity and thus the complexity of programs run on the opto-plate 10.

Continuing with the implementation of FIG. 2A, an output port 44 is disposed on the base 12, for the outputting of the data signal used for updating the LED driver 30 settings, as would be understood by one of skill in the art. It is understood that data routed through this output port 44 can facilitate daisy-chaining multiple boards together that are driven by a single microcontroller 34 if desired.

FIG. 2B is a close-up view of the opto-plate 10 showing the connection of a current-sink LED driver 30 to the individual LED light fixtures 20A, 20B. In FIG. 2B, the driver footprint 30A, as well as the two-channel 22A, 22B and one-channel 24A, 24B LED footprints are shown. In various implementations, each driver channel 44A, 44B is in electrical communication through a via 45A to one of the one-channel 48A or two-channel 48B, 48C cathodes of each LED fixture 20A, 20B, respectively. It is understood that each of the vias 45A, 45B, 45C is in electrical communication with cathodes on the top layer and the driver channels on various layers of the board (not shown). In these implementations, the anodes 46A, 46B, 46C are in electrical communication with the power source 18, as is shown in FIG. 1. Alternate configurations are of course possible.

Turning to FIGS. 3A-B, in exemplary implementations as discussed above, adaptors 14 are fitted between the opto-plate 10 and a cell culture plate (not shown). In various implementations, these adaptors 14 allow for independent, diffuse illumination of individual culture wells with no well-to-well bleed-through. One such adaptor 14 embodiment is shown in FIGS. 3A and 3B.

In the implementations of FIGS. 3A-B, the adaptor 14 has three layers: top 50, middle 52 and bottom 54, each comprising openings 60A, 60B, 60C corresponding to an individual well in the associated plate (not shown) to be positioned thereon.

In this implementation, in the middle 52 and bottom 54 layers, each opening 60B, 60C in the layers 52, 54 is surrounded by walls 62A, 62B, 64A, 64B that interlock with complementary walls on the above layer, thereby ensuring that light from one well corresponding to one opening 61 cannot spread to a neighboring well corresponding to another opening 60.

In this implementation, diffuser paper (not shown), such as two 8 mm×8 mm squares of paper can be placed within the layers defining the openings 60A, 60B, 60C of each of the wells 60. In these implementations, the paper can be disposed between the layers at each well opening 60, so as to be locked into place when the layers 50, 52, 54 are locked in place. In these implementations, two layers of diffuser paper for each well opening 60 (one in the space between each adapter layer) can facilitate good diffusion of LED light, important for each cell receiving comparable amount of light. In various implementations, the layers 50, 52, 54 have mounting points 70, 72, 74, 76, 78 such that fasteners can be passed through the openings to 70, 72, 74, 76, 78 attach the layers and affix the adaptor to the base 12 at the opto-plate openings (shown in FIG. 2A at 28). It is further understood that threaded press-fit inserts can be used in certain implementations.

Continuing with FIGS. 3A-B, the top layer 50 is configured to fit dimensions of the underside of the 96-well plate (not shown) being used, so that the plate sits flush on top of the adapter face 14A. Accordingly, in certain implementations, the adapter face 14A has a mounting ridge 80 disposed on the top layer 50, as would be understood.

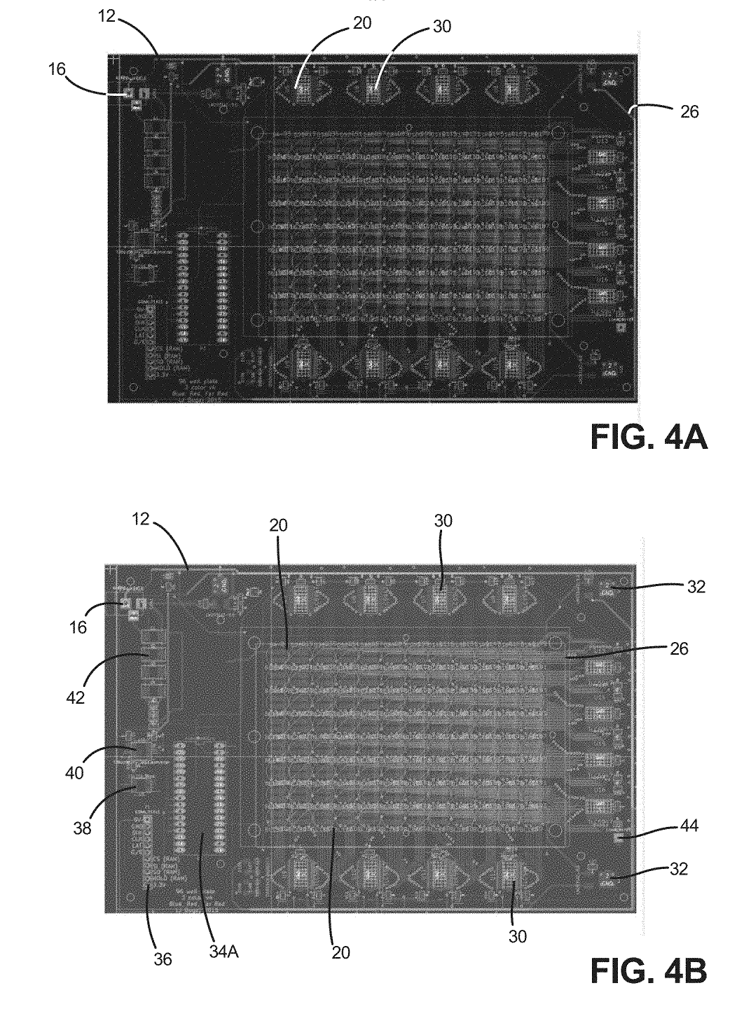

FIGS. 4A-4B depict further views of the opto-plate 10, according to certain implementations. In these implementations, the base 12 has several LED fixtures 20 disposed on the LED board 26 similar to the fixtures 20 discussed above and powered by the LED drivers 30, transistors 33, microcontroller mounting header 34A, memory 38, and logic converter 40 as described above in relation to FIG. 2A. In these implementations, additional pin headers 36, and the output port 44 are also shown. Surface mount pads 42 for additional capacitors can also be provided.

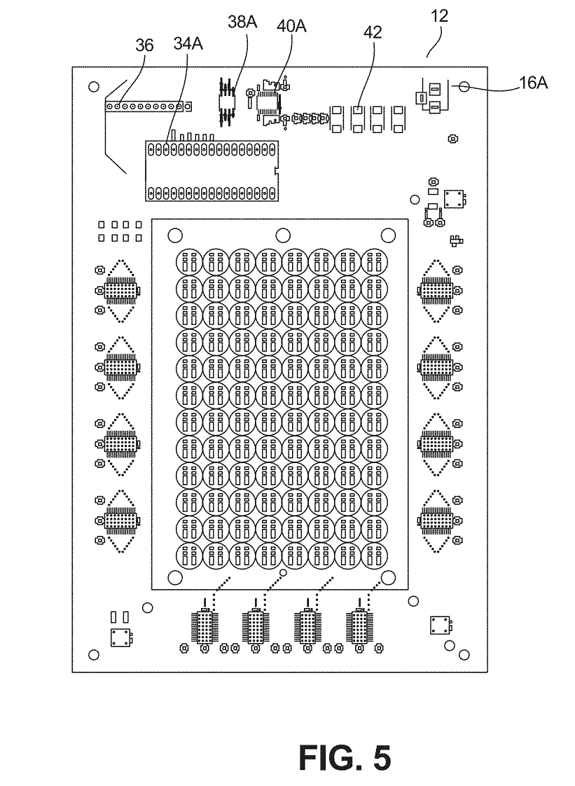

Additionally, FIG. 5 depicts the mounting points or headers for the microcontroller 34A, the memory 38A, the logic converter 40A and the power jack 16A, according to one embodiment.

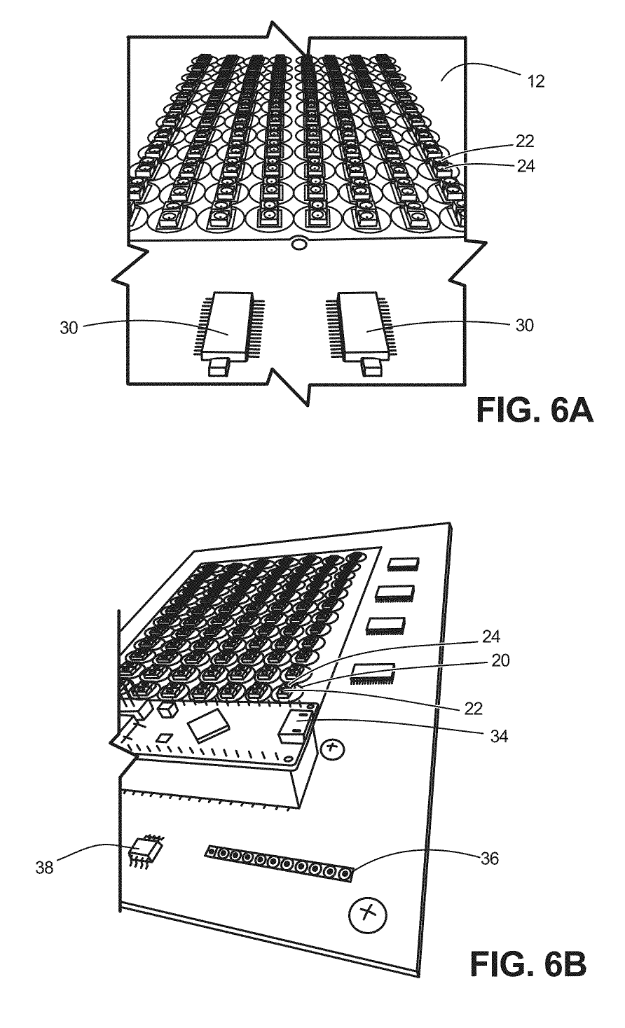

The implementations of FIGS. 6A-B depict various opto-plate components installed on the base 12. For example, these figures depict 2-color LED 22 and 1-color LED 24 channels mounted in the LED fixtures 20, the microcontroller 34 and drivers 30. It is understood that various alternate implementations are possible.

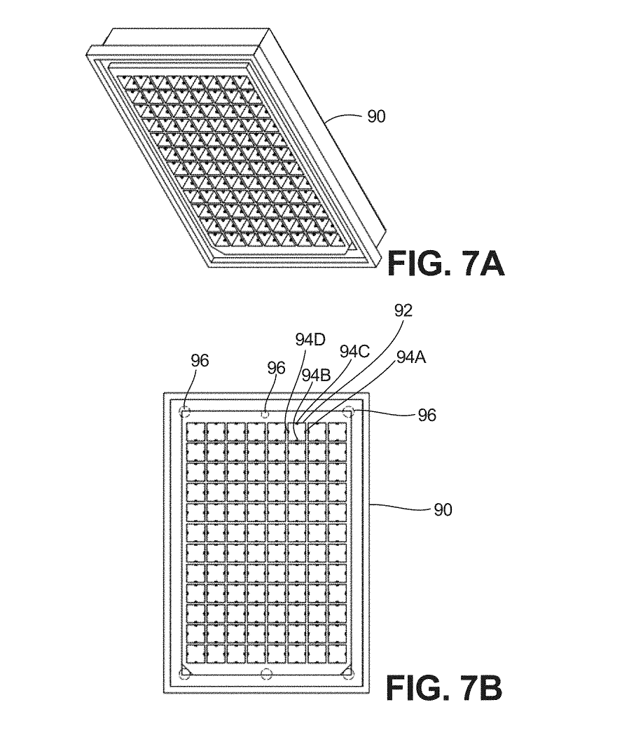

FIGS. 7A-B depict an embodiment of a 384-well plate adaptor 90. That is, the adaptor 90 can be used with a 384-well culture plate (not shown). In this implementation, the 384-well plate adaptor 90 can be single-layered or have several layers and be mounted to a base 12 in a fashion similar to that described in relation to the 96-well adaptor of FIGS. 3A-B. In this implementation, however, each opening 92 in the adaptor 90 is configured to illuminate a group of four wells of the 384-well plate, as would be understood by one of skill in the art. Further, in this implementation, each well opening 92 has diffuser protrusions 94A, 94B, 94C, 94D radially disposed about the edge of the opening 92 to support a known diffuser, such as diffuser paper (not shown). Several mounting points 96 are also disposed about the adaptor 90 for attachment of the adaptor 90 to an opto-plate embodiment, such as opto-plate 10 as was described above in relation to FIGS. 3A-B.

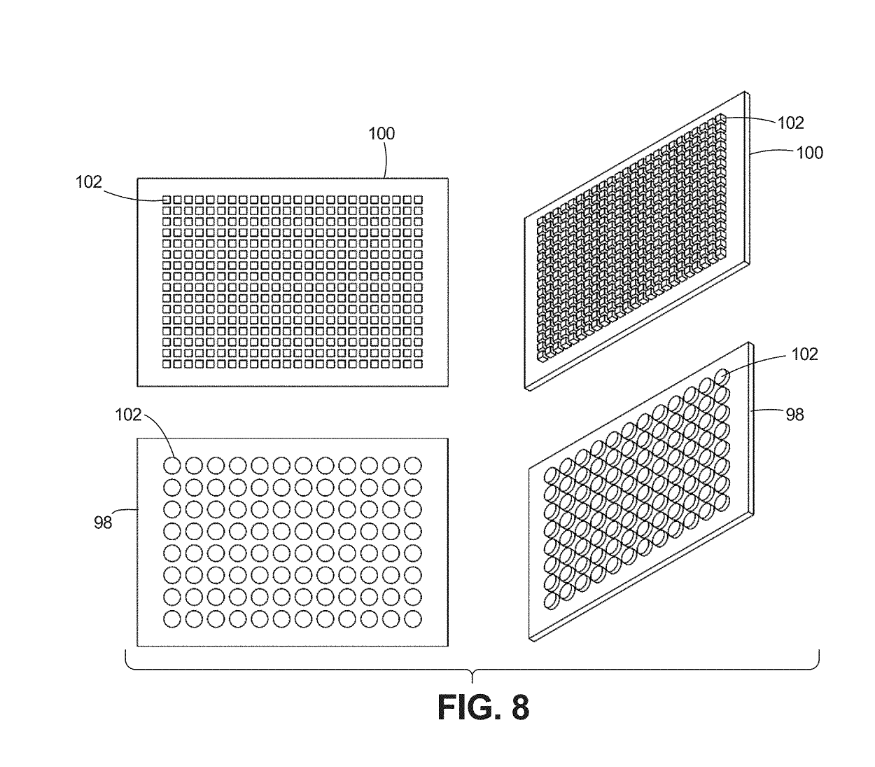

In FIG. 8, a 96-well lid 98 and 384-well lid 100 are shown. To prevent light bleed-through between the individual wells of the plate (not shown), such as that caused by reflection of light within an incubator, these lids 98, 100 can be used in conjunction with 96- and 384-well plates (not shown), respectively. That is, each lid 98, 100 can be placed on a corresponding well plate. In various implementations, the lids 98, 100 have well-shaped inserts 102 that fit into each well (not shown) to guide the lid 98, 100 into place and further prevent light reflection and contamination of neighboring wells of the plates (not shown).

Although the disclosure has been described with reference to preferred embodiments, persons skilled in the art will recognize that changes may be made in form and detail without departing from the spirit and scope of the disclosed apparatus, systems and methods.

### Example

One exemplary use of the illumination opto-plate 10 is found in “Cancer Mutations and Targeted Drugs Can Disrupt Dynamic Signal Encoding by the Ras/Erk Pathway.” by Bugaj, L. J., Sabnis, A., Mitchell, A., Garbarino, J., Toettcher, J. E., Bivona, T. G., and Lim, W. A. and is hereby incorporated by reference in its entirety for all purposes.

For more high-throughput and long-term analysis, the optoPlate 12, a device for optogenetic illumination in microwell plates (FIG. 1) was developed. This device allowed for stimulation of cells dynamically across a large parameter space and analysis of multiple cellular outputs over time through fixed-cell fluorescence microscopy.

To test if changes in signal transmission dynamics could alter gene expression decisions in cells, the levels of several downstream output proteins in response to optoSOS inputs were measured. An experimental model was designed to isolate the effects of altered Ras/Erk kinetics in a well-controlled cell line lacking potentially confounding mutations. Thus, the responses of wild-type NIH 3T3 cells in the presence and absence of 100 μM SB590885 (paradox inhibitor of B-Raf) could be compared. This concentration of drug extended Ras/Erk pathway decay kinetics while minimally elevating basal ppErk levels. Cells were seeded and starved in 384-well plates, and, in the presence or absence of drug, were exposed to various dynamic input patterns using the optoPlate 12. After stimulating the cells over several hours, cells were fixed and immunostained for Erk-dependent transcriptional targets.

For all optogenetic experiments, cells were supplemented with HPLC-purified phycocyanobilin (PCB, Frontier Scientific #P14137) at a concentration of 5 uM (3T3s) or 10 uM (all other cells). Cells were incubated in PCB for ˜0.5-1 hr before optogenetic stimulation. For bulk Western blot experiments, cells were illuminated in a cell culture incubator with a custom built panel of either 650 nm or 750 nm LEDs for activation or inactivation of optoSOS, respectively. For 96- and 384-well In-Cell Western and immunofluorescence experiments, optogenetic experiments were performed with a custom-built 96-well “optoplate” 10 with adapters 14 accommodating either 96- or 384-well plates (FIGS. 1, 2, and 5). Briefly, a printed circuit board was designed using the Kicad software package and manufactured through PCBUnlimited (PCBUnlimited.com). The circuit board design allowed placement of 192 independently addressable LEDs 20, with two LEDs 20—one red (Vishay, VLMK31R1S2-GS18), one far-red (Marubeni, SMT780)—fitting under each well position. The LEDs 20 shared a common anode 46A-C, and each cathode was connected to one of 12 24-channel 44A-B constant-current LED drivers 30 (TLC5947, Texas Instruments). These drivers 30 allow independent 12-bit grayscale control (0-4095) of each LED 20 using pulse-width modulation. These LED drivers 30 were controlled by an on-board Arduino Micro microcontroller 34, which was programmed with custom script through the Arduino IDE. Custom adapters 14 interfacing with 96- and 384-well plates were designed in the Autodesk Inventor program and printed on a Stratasys uPrint 3D printer.

The optoPlate 12 was designed to enable incubator-compatible optogenetic illumination allowing independent control of all wells in microwell plates. The optoPlate 12 is designed to independently illuminate 96 well positions corresponding to the wells of a 96-well plate. This device can also be adapted for use with 384 well plates, as each position in a standard 96-well plate corresponds exactly to 4 wells of a 384 well plate. While those 4 wells will receive the same light input, culture conditions or downstream immunostaining can be varied, expanding the parameter space for an individual experiment.

Though the optoPlate 12 is designed to illuminate up to three colors per position, in this work only two (red and far-red) were used. Illumination is driven by an Arduino Micro microcontroller 34 communicating with 12 onboard LED driver 30 chips (TLC5947). Illumination parameters are specified through a custom script written in the Arduino IDE where illumination intensity and timing can be defined for each LED 20 in each position. The wiring diagram of the circuitboard is depicted (FIG. 4A), while the assembled optoPlate 12 is shown in FIG. 2A. Each position holds two LEDs 20 positioned underneath 1 96-well well or 4 384-well wells. The fully assembled optoPlate 12 (FIG. 1) is mounted with a 3D printed adapter 14, which prevents well-to-well bleedthrough. The schematic for the optoPlate 12 adapter 14 in FIGS. 7A and 7B is designed for both 96- and 384-well plates.

What is claimed is:

1. An illumination device, comprising:
a. a base, comprising;
i. a plurality of LED fixtures;
ii. a microcontroller; and
iii. at least one LED driver; and
b. an adaptor, comprising;
i. at least one layer;
ii. a plurality of openings; and
iii. a plurality of walls,
wherein the isled fixtures are configured to illuminate a culture plate.

2. The illumination device of claim 1, wherein each of the plurality of LED fixtures is independently programmable.

3. The illumination device of claim 1, wherein the adaptor has three layers.

4. The illumination device of claim 3, wherein a plurality of diffuser paper is disposed between the adaptor layers.

5. The illumination device of claim 3, wherein the adaptor layers further comprise mounting points.

6. The illumination device of claim 3, wherein the adaptor further comprises a mounting ridge configured for reception of the culture plate.

7. The illumination device of claim 6, wherein the walls of the adaptor are configured for individual well illumination.

8. The illumination device of claim 7, wherein the opto-plate further comprises a memory.

9. A system for illuminating a culture plate comprising:
a. an opto-plate, comprising:
i. at least one LED fixture;
ii. at least one LED driver; and
iii. at least one microcontroller;
b. an adaptor, comprising;
i. at least one layer;
ii. at least one opening; and
iii. a plurality of walls, wherein the adaptor is attached to the opto-plate such that when the culture plate is placed on the adaptor the LED fixtures illuminate the culture plate.

10. The system of claim 9, wherein the walls of the adaptor are configured such that each well within the culture plate is individually illuminated.

11. The system of claim 9, further comprising a lid.

12. The system of claim 9, further comprising an Arduino configured to run independent of a computer.

13. The system of claim 12, further comprising an output port configured for linkage of multiple opto-plates.

14. The system of claim 9, further comprising a diffuser.

15. An illumination device, comprising:
a. an opto-plate, comprising at least one LED fixture; and
b. an adaptor,
wherein the illumination device is configured to illuminate a culture plate.

16. The illumination device of claim 15, wherein the opto-plate further comprises:
a. at least one LED driver;
b. at least one microcontroller;
c. a power jack; and
d. an output port.

17. The illumination device of claim 16, wherein the opto-plate has at least one of the following features:
a. a memory;
b. a logic converter;
c. a capacitor; and
d. a resistor.

18. The illumination device of claim 17, the adaptor further comprising;
a. a mounting ridge, configured to hold a culture plate; and
b. a lid.

19. The illumination device of claim 15, configured to illuminate a 96 well culture plate.

20. The illumination device of claim 15, configured to illuminate a 384 well culture plate.
